SEQUENCE LISTING (10 sequences)

SEQ ID NO: 1 - DNA, 1878 bp, Mus musculus
  CDS (1)..(1875)
atggccccgcgcgcccggcggcgccgccagctgcccgcgccgctgctggcgctctgcgtgctgctcgttccactgcaggtgactctccaggtcactcctccatgcacccaggagaggcattatgagcatctcggacggtgttgcagcagatgcgaaccaggaaagtacctgtcctctaagtgcactcctacctccgacagtgtgtgtctgccctgtggccccgatgagtacttggacacctggaatgaagaagataaatgcttgctgcataaagtctgtgatgcaggcaaggccctggtggcggtggatcctggcaaccacacggccccgcgtcgctgtgcttgcacggctggctaccactggaactcagactgcgagtgctgccgcaggaacacggagtgtgcacctggcttcggagctcagcatcccttgcagctcaacaaggatacggtgtgcacaccctgcctcctgggcttcttctcagatgtcttttcgtccacagacaaatgcaaaccttggaccaactgcaccctccttggaaagctagaagcacaccaggggacaacggaatcagatgtggtctgcagctcttccatgacactgaggagaccacccaaggaggcccaggcttacctgcccagtctcatcgttctgctcctcttcatctctgtggtagtagtggctgccatcatcttcggcgtttactacaggaagggagggaaagcgctgacagctaatttgtggaattgggtcaatgatgcttgcagtagtctaagtggaaataaggagtcctcaggggaccgttgtgctggttcccactcggcaacctccagtcagcaagaagtgtgtgaaggtatcttactaatgactcgggaggagaagatggttccagaagacggtgctggagtctgtgggcctgtgtgtgcggcaggtgggccctgggcagaagtcagagattctaggacgttcacactggtcagcgaggttgagacgcaaggagacctctcgaggaagattcccacagaggatgagtacacggaccggccctcgcagccttcgactggttcactgctcctaatccagcagggaagcaaatctatacccccattccaggagcccctggaagtgggggagaacgacagtttaagccagtgtttcaccgggactgaaagcacggtggattctgagggctgtgacttcactgagcctccgagcagaactgactctatgcccgtgtcccctgaaaagcacctgacaaaagaaatagaaggtgacagttgcctcccctgggtggtcagctccaactcaacagatggctacacaggcagtgggaacactcctggggaggaccatgaaccctttccagggtccctgaaatgtggaccattgccccagtgtgcctacagcatgggctttcccagtgaagcagcagccagcatggcagaggcgggagtacggccccaggacagggctgatgagaggggagcctcagggtccgggagctcccccagtgaccagccacctgcctctgggaacgtgactggaaacagtaactccacgttcatctctagcgggcaggtgatgaacttcaagggtgacatcatcgtggtgtatgtcagccagacctcgcaggagggcccgggttccgcagagcccgagtcggagcccgtgggccgccctgtgcaggaggagacgctggcacacagagactcctttgcgggcaccgcgccgcgcttccccgacgtctgtgccaccggggctgggctgcaggagcagggggcaccccggcagaaggacgggacatcgcggccggtgcaggagcagggtggggcgcagacttcactccatacccaggggtccggacaatgtgcagaatga

SEQ ID NO: 2 - protein, 625 aa, Mus musculus
MAPRARRRRQLPAPLLALCVLLVPLQVTLQVTPPCTQERHYEHLGRCCSRCEPGKYLSSKCTPTSDSVCLPCGPDEYLDTWNEEDKCLLHKVCDAGKALVAVDPGNHTAPRRCACTAGYHWNSDCECCRRNTECAPGFGAQHPLQLNKDTVCTPCLLGFFSDVFSSTDKCKPWTNCTLLGKLEAHQGTTESDVVCSSSMTLRRPPKEAQAYLPSLIVLLLFISVVVVAAIIFGVYYRKGGKALTANLWNWVNDACSSLSGNKESSGDRCAGSHSATSSQQEVCEGILLMTREEKMVPEDGAGVCGPVCAAGGPWAEVRDSRTFTLVSEVETQGDLSRKIPTEDEYTDRPSQPSTGSLLLIQQGSKSIPPFQEPLEVGENDSLSQCFTGTESTVDSEGCDFTEPPSRTDSMPVSPEKHLTKEIEGDSCLPWVVSSNSTDGYTGSGNTPGEDHEPFPGSLKCGPLPQCAYSMGFPSEAAASMAEAGVRPQDRADERGASGSGSSPSDQPPASGNVTGNSNSTFISSGQVMNFKGDIIVVYVSQTSQEGPGSAEPESEPVGRPVQEETLAHRDSFAGTAPRFPDVCATGAGLQEQGAPRQKDGTSRPVQEQGGAQTSLHTQGSGQCAE

SEQ ID NO: 3 - DNA, 1851 bp, Homo sapiens
  CDS (1)..(1851)
atggccccgcgcgcccggcggcgccgcccgctgttcgcgctgctgctgctctgcgcgctgctcgcccggctgcaggtggctttgcagatcgctcctccatgtaccagtgagaagcattatgagcatctgggacggtgctgtaacaaatgtgaaccaggaaagtacatgtcttctaaatgcactactacctctgacagtgtatgtctgccctgtggcccggatgaatacttggatagctggaatgaagaagataaatgcttgctgcataaagtttgtgatacaggcaaggccctggtggccgtggtcgccggcaacagcacgaccccccggcgctgcgcgtgcacggctgggtaccactggagccaggactgcgagtgctgccgccgcaacaccgagtgcgcgccgggcctgggcgcccagcacccgttgcagctcaacaaggacacagtgtgcaaaccttgccttgcaggctacttctctgatgccttttcctccacggacaaatgcagaccctggaccaactgtaccttccttggaaagagagtagaacatcatgggacagagaaatccgatgcggtttgcagttcttctctgccagctagaaaaccaccaaatgaaccccatgtttacttgcccggtttaataattctgcttctcttcgcgtctgtggccctggtggctgccatcatctttggcgtttgctataggaaaaaagggaaagcactcacagctaatttgtggcactggatcaatgaggcttgtggccgcctaagtggagataaggagtcctcaggtgacagttgtgtcagtacacacacggcaaactttggtcagcagggagcatgtgaaggtgtcttactgctgactctggaggagaagacatttccagaagatatgtgctacccagatcaaggtggtgtctgtcagggcacgtgtgtaggaggtggtccctacgcacaaggcgaagatgccaggatgctctcattggtcagcaagaccgagatagaggaagacagcttcagacagatgcccacagaagatgaatacatggacaggccctcccagcccacagaccagttactgttcctcactgagcctggaagcaaatccacacctcctttctctgaacccctggaggtgggggagaatgacagtttaagccagtgcttcacggggacacagagcacagtgggttcagaaagctgcaactgcactgagcccctgtgcaggactgattggactcccatgtcctctgaaaactacttgcaaaaagaggtggacagtggccattgcccgcactgggcagccagccccagccccaactgggcagatgtctgcacaggctgccggaaccctcctggggaggactgtgaacccctcgtgggttccccaaaacgtggacccttgccccagtgcgcctatggcatgggccttccccctgaagaagaagccagcaggacggaggccagagaccagcccgaggatggggctgatgggaggctcccaagctcagcgagggcaggtgccgggtctggaagctcccctggtggccagtcccctgcatctggaaatgtgactggaaacagtaactccacgttcatctccagcgggcaggtgatgaacttcaagggcgacatcatcgtggtctacgtcagccagacctcgcaggagggcgcggcggcggctgcggagcccatgggccgcccggtgcaggaggagaccctggcgcgccgagactccttcgcggggaacggcccgcgcttcccggacccgtgcggcggccccgaggggctgcgggagccggagaaggcctcgaggccggtgcaggagcaaggcggggccaaggcttga

SEQ ID NO: 4 - protein, 616 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 5 - protein, 443 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGRSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 6 aa, Artificial sequence
  Peptide
HHHHHH

SEQ ID NO: 7 - DNA, 1356 bp, Homo sapiens
  CDS (95)..(1297)
gtatatataacgtgatgagcgtacgggtgcggagacgcaccggagcgctcgcccagccgccgyctccaagcccctgaggtttccggggaccacaatgaacaagttgctgtgctgcgcgctcgtgtttctggacatctccattaagtggaccacccaggaaacgtttcctccaaagtaccttcattatgacgaagaaacctctcatcagctgttgtgtgacaaatgtcctcctggtacctacctaaaacaacactgtacagcaaagtggaagaccgtgtgcgccccttgccctgaccactactacacagacagctggcacaccagtgacgagtgtctatactgcagccccgtgtgcaaggagctgcagtacgtcaagcaggagtgcaatcgcacccacaaccgcgtgtgcgaatgcaaggaagggcgctaccttgagatagagttctgcttgaaacataggagctgccctcctggatttggagtggtgcaagctggaaccccagagcgaaatacagtttgcaaaagatgtccagatgggttcttctcaaatgagacgtcatctaaagcaccctgtagaaaacacacaaattgcagtgtctttggtctcctgctaactcagaaaggaaatgcaacacacgacaacatatgttccggaaacagtgaatcaactcaaaaatgtggaatagatgttaccctgtgtgaggaggcattcttcaggtttgctgttcctacaaagtttacgcctaactggcttagtgtcttggtagacaatttgcctggcaccaaagtaaacgcagagagtgtagagaggataaaacggcaacacagctcacaagaacagactttccagctgctgaagttatggaaacatcaaaacaaagcccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggcgaataaaaaatggcgaccaagacaccttgaagggcctaatgcacgcactaaagcactcaaagacgtaccactttcccaaaactgtcactcagagtctaaagaagaccatcaggttccttcacagcttcacaatgtacaaattgtatcagaagttatttttagaaatgataggtaaccaggtccaatcagtaaaaataagctgcttataactggaaatggccattgagctgtttcctcacaattggcgagatcccatggatgataa

SEQ ID NO: 8 - protein, 401 aa, Homo sapiens
MNKLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKAQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 9 - DNA, 954 bp, Homo sapiens
  CDS (1)..(951)
atgcgccgcgccagcagagactacaccaagtacctgcgtggctcggaggagatgggcggcggccccggagccccgcacgagggccccctgcacgccccgccgccgcctgcgccgcaccagccccccgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattga

SEQ ID NO: 10 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

(19) United States
(12) Patent Application Publication
(10) Pub. No.: US 2003/0017151 A1
(43) Pub. Date: Jan. 23, 2003

(54) Therapeutic use of rank antagonists

(76) Inventors: William C. Dougall, Seattle, WA (US); Dirk M. Anderson, Seattle, WA (US)

Correspondence Address: IMMUNEX CORPORATION, LAW DEPARTMENT, 51 UNIVERSITY STREET, SEATTLE, WA, 98101

(21) Appl. No.: 10/151,071

(22) Filed: May 17, 2002

Related U.S. Application Data
(60) Provisional application No. 60/291,919, filed on May 17, 2001.

(51) Int. Cl. A61K 48/00; A61K 39/395

(52) U.S. Cl. 424/143.1; 514/44

(57) ABSTRACT

Provided herein are methods of treating various medical conditions that require the formation of new bone by administering an effective amount of a RANK antagonist. Antagonists suitable for use in the subject methods include soluble RANK polypeptides that bind RANKL, antisense oligonucleotides that inhibit RANK activity, antibodies specific for RANK or RANKL and osteoprotegerin.

This patent application claims the benefit of priority under 35 U.S.C. §119 to U.S. Provisional Application Serial No. 60/291,919, the disclosure of which is incorporated herein by reference.

### TECHNICAL FIELD OF THE INVENTION

The present invention relates generally to the therapeutic use of antagonists of the RANK/RANKL interaction in medical conditions that require the formation of new bone.

### BACKGROUND OF THE INVENTION

RANK (Receptor Activator of NF-κB) and its ligand (RANKL) are a receptor/ligand pair that play an important role in immune responses and in bone metabolism. RANK and RANKL, both murine and human, have been cloned and characterized (see, for example, U.S. Pat. No. 6,017,729, WO 98/25958, EP 0 873 998, EP 0 911 342, U.S. Pat. No. 5,843,678, WO 98/46751 and WO 98/54201).

It has been shown that RANKL binds not only to RANK, but also to a naturally occurring RANK decoy protein called osteoprotegerin (OPG), which is a member of the tumor necrosis factor receptor family (see, for example, U.S. Pat. No. 6,015,938 and WO 98/46751). OPG is a soluble molecule whose role in bone metabolism is reviewed in Hofbauer et al., J Bone Min Res 15(1):2-12 (2000). Further aspects of RANK/RANKL and OPG biology are discussed, for example, in Simonet et al., Cell 89:309-319 (1997); Kodaira et al., Gene 230:121-27 (1999); U.S. Pat. No. 5,843,678; and U.S. Pat. No. 6,015,938. In contrast to RANK, OPG also binds a second binding partner, which is known as “TNF-related apoptosis inducing ligand,” or “TRAIL.” TRAIL induces apoptosis in a wide variety of transformed human cell lines in vitro, and is being tested for its therapeutic potential in treating human tumors. OPG acts to suppress RANK activity by binding to RANKL, thereby preventing it from binding RANK, and has been proposed as a therapeutic agent for a variety of conditions that are characterized by bone loss (WO 98/46751; WO 01/03719; WO 01/16299; WO 01/17543; and WO 01/03719).

RANK, a Type I transmembrane protein, is a member of the TNF receptor superfamily (see, for example, U.S. Pat. No. 6,017,729). Full-length human RANK polypeptide has 616 amino acids. Human RANKL is a 317 amino acid protein of the tumor necrosis factor ligand family, and is a type II membrane protein lacking a signal peptide and having a short cytoplasmic domain and an extracellular region that binds specifically with RANK (see, for example, U.S. Pat. No. 6,017,729). RANKL also has been called “osteoprotegerin binding protein,” “osteoclastogenesis differentiation factor,” and “TRANCE” (see, for example, Kodaira et al., 1999; Yasuda et al., Proc. Natl. Acad. Sci. 95:3597 (1998); and Wong et al., J Biol Chem 273(43):28355-59 (1998)).

The RANK protein instigates intracellular events by interacting with various TNF Receptor Associated Factors (TRAFs) (see, for example, Galibert et al., J Biol Chem 273(51):34120-27 (1998); Damay et al., J Biol Chem 273(32):20551-55 (1998); and Wong et al., 1998). The triggering of RANK, such as by its interaction with its ligand RANKL, activates TRAF-mediated intracellular events that result in the upregulation of the transcription factor NF-κB, a ubiquitous transcription factor that is extensively utilized in cells of the immune system

RANK is expressed primarily on the surface of epithelial cells, some B and T cell lines, fibroblasts, dendritic cells and osteoclasts and their precursors. RANKL, which also exists in a soluble form, is expressed primarily in hematopoietic tissues, such as bone marrow, thymus and spleen, and including T cells and osteoblast lineage cells. Signals mediated by the RANK/RANKL interaction are involved in stimulating the differentiation and function of osteoclasts, the cells responsible for bone resorption (see, for example, Lacey et al., Cell 93:165-76 (1998); Yasuda et al., 1998)). This process appears to involve direct contact between cells expressing RANKL and osteoclast precursors. Accordingly, it has been proposed that osteoprotegerin or soluble forms of RANK that block RANKL binding could be administered to inhibit osteoclast activity, thereby slowing the rate of bone loss associated with osteoporosis, hypercalcemia of malignancy, rheumatoid arthritis, prosthetic loosening and so on (see, for example, WO 98/46751, WO 99/58674, WO 01/16299 and Hofbauer et al., 2000).

Several investigators have reported on the in vivo effects of RANK antagonists that are derived from the RANK protein (see, for example, U.S. Pat. No. 6,015,938 and WO 98/46751). Others have reported that administration of soluble RANK reduced bone destruction in mouse models of human disease (see Oyajobi et al., J Bone Min Res 15 (suppl. 1):S176, Abstract #1151 (September, 2000); Oyajobi et al., Cancer Res 61:2572-78 (2001); Childs et al., Abstract, Orthopedic Research Society, San Francisco, 2001).

Therapeutic approaches to the treatment of bone diseases have improved greatly in recent years (see Rodan and Martin, Science 289:1508-1514 (2000)). For example, peptides have been identified that promote bone deposition (WO 00/75185). However, there remains a need to provide better therapies for various medical conditions in which formation of new bone is desirable.

### SUMMARY OF THE INVENTION

Provided herein are methods and compositions for treating various medical conditions that require the formation of new bone. The subject therapies and compositions can be administered to stimulate bone formation in patients who are not actively losing bone. Patients who will benefit from these therapies include those who formerly suffered a loss of bone density but who are not currently experiencing any bone loss. Patients who will benefit from these treatments include those whose condition is not characterized by loss of bone density, but who nonetheless require new bone formation, such as for example, accident victims who have lost bone due to traumatic injury.

RANK antagonists suitable for use in the subject methods and compositions include: an antibody capable of specifically binding RANK and that does not trigger RANK; an antibody capable of specifically binding RANKL; an antisense oligonucleotide that blocks translation or transcription of RANK or RANKL mRNA; an osteoprotegerin polypeptide; and a soluble RANK polypeptide that is capable of binding RANKL. Soluble RANK proteins useful as RANK antagonists will comprise a RANKL-binding portion of the extracellular region of a RANK polypeptide, including allelic variants and muteins so long as they retain the ability to bind RANKL.

Specific embodiments of the invention include the following.

Provided here are methods of treatment that involve administering a RANK antagonist to a patient having one of the following medical conditions: acute septic arthritis, osteomalacia (including ricketts and scurvy), hyperparathyroidism, Cushing's syndrome, monoostotic fibrous dysplasia, polyostotic fibrous dysplasia, Gaucher's disease, Langerhans' cell histiocytosis, spinal cord injury, patients requiring periodontal reconstruction or patients who have completed a course of radiation therapy for cancer. The RANK antagonist is administered in an amount and at a frequency sufficient to stimulate an increase in the rate of formation of new bone in the patient. The RANK antagonist used for this method is one that is capable of inhibiting the ability of a RANK protein to induce NF-κB, wherein RANK is a protein consisting of amino acids 1-616 of SEQ ID NO:4, and wherein the RANK antagonist is one of the following: an antibody capable of specifically binding a RANK polypeptide comprising amino acids 33-196 of SEQ ID NO:4; an antisense oligonucleotide that blocks transcription or translation of RANK or RANKL mRNA; an antibody capable of specifically binding a RANKL polypeptide comprising amino acids 69-317 of SEQ ID NO:10; or an OPG polypeptide that comprises amino acids 22-185 of SEQ ID NO:8 and is capable of binding a RANKL polypeptide consisting of amino acids 1-317 of SEQ ID NO:10. In one embodiment of this method, the patient has experienced no loss of bone density for at least one month preceding the initiation of treatment. During administration of the RANK antagonist, the sufficiency of treatment may be monitored by repeatedly measuring bone density during the time the treatment is being administered. One of the RANK antagonists suitable for use in this method is an antibody capable of specifically binding a RANKL polypeptide comprising amino acids 69-317 of SEQ ID NO:10, which is the extracellular domain of human RANKL. Also suitable for use as a RANK antagonist is an OPG polypeptide that comprises amino acids 22-185 of SEQ ID NO:8 and is capable of binding a RANKL polypeptide consisting of amino acids 1-317 of SEQ ID NO:10.

Another embodiment of the invention provides methods of treating a patient who has acute septic arthritis, osteomalacia, hyperparathyroidism, Cushing's syndrome, monoostotic fibrous dysplasia, polyostotic fibrous dysplasia, Gaucher's disease, Langerhans' cell histiocytosis, spinal cord injury, patients requiring periodontal reconstruction or who has completed a course of radiation therapy. For this method, the patient is administered a RANK antagonist in an amount and at a frequency sufficient to stimulate an increase in the rate of formation of new bone. The RANK antagonist to be used here is one that is capable of inhibiting the ability of RANK to induce NF-κB, wherein RANK is a protein consisting of amino acids 1-616 of SEQ ID NO:4. For this method, the RANK antagonist is a soluble RANK polypeptide that is capable of binding a RANKL polypeptide comprising amino acids 69-317 of SEQ ID NO:10, and where the soluble RANK polypeptide has an at least 90% identity to a RANK polypeptide comprising amino acids 33-196 of SEQ ID NO:4. In one embodiment of this invention, the patient has not experienced loss of bone density for at least one month preceding the initiation of treatment. In another aspect of this method, the soluble RANK polypeptide is encoded by a nucleic acid molecule that is capable of hybridizing under stringent conditions with a nucleic acid molecule consisting of the nucleotide sequence shown in SEQ ID NO:3 or its complement, where the stringent conditions comprise hybridizing in 6×SSC at 63° C., and washing in 3×SSC at 55° C. In one embodiment, the soluble RANK polypeptide comprises amino acids 33-196 of SEQ ID NO:10. In yet another embodiment, the soluble RANK polypeptide further comprises a moiety selected from the group consisting of an immunoglobulin Fc domain, a FLAG™ tag, a peptide comprising at least about 6 His residues, a leucine zipper, polyethylene glycol and combinations thereof. In one embodiment, the soluble RANK polypeptide is linked covalently to an immunoglobulin Fc domain. In one aspect of the invention, the RANK antagonist is a RANK:Fc fusion protein consisting of amino acids 30-433 of SEQ ID NO:5, or alternatively, is a variant of this fusion protein in which glutamic acid is substituted for aspartic acid at residue 352 and methionine is substituted for leucine at residue 354. The sufficiency of treatment may be monitored by repeatedly measuring bone density during the time the treatment is being administered.

Also provided herein are methods of treating a patient who is a prosthetic joint recipient, a bone graft recipient or a ligament graft recipient by administering to the patient a RANK antagonist in an amount and at a frequency sufficient to stimulate an increase in the rate of formation of new bone, where the RANK antagonist is one of the following: a soluble RANK polypeptide that is capable of binding a RANKL polypeptide comprising amino acids 69-317 of SEQ ID NO:10, where the soluble RANK polypeptide has an at least 90% identity to a RANK protein comprising amino acids 33-196 of SEQ ID NO:4; an antibody capable of specifically binding a RANK polypeptide comprising amino acids 33-196 of SEQ ID NO:4; an antisense oligonucleotide that blocks transcription or translation of RANK or RANKL mRNA; an antibody capable of specifically binding a RANKL polypeptide comprising amino acids 69-317 of SEQ ID NO:10; or an OPG polypeptide that comprises amino acids 22-185 of SEQ ID NO:8 and is capable of binding a RANKL polypeptide consisting of amino acids 1-317 of SEQ ID NO:10. For example, the RANK antagonist may be a soluble RANK polypeptide comprising amino acids 33-196 of SEQ ID NO:4. When a soluble RANK polypeptide is used as the RANK antagonist, the antagonist protein may further comprise another moiety which is an immunoglobulin Fc domain, a FLAG™ tag, a peptide comprising at least about 6 His residues, a leucine zipper, polyethylene glycol or combinations thereof. A suitable RANK:Fc fusion protein for use as described here is one consisting of the amino acid sequence shown in SEQ ID NO:5, or a variant of this protein in which glutamic acid is substituted for asparatic acid at residue 352 and methionine is substituted for leucine at residue 354. In treating these patients, the first dose of the antagonist is administered within one month of surgical implantation of the prosthetic joint, bone graft or ligament graft. In one embodiment of this method, the sufficiency of treatment is monitored by repeatedly measuring bone density during the time the treatment is being administered.

### DETAILED DESCRIPTION OF THE INVENTION

The invention provides methods and compositions for treating medical conditions characterized by the need for formation of new bone. Preferably, the patient is a human, but the subject methods may be applied to any mammal, including domestic animals such as pets and farm animals. “Formation of new bone,” as used herein, means a net increase in the amount of hard calcified bone tissue in one or more of the patient's bones. The subject methods involve administering to a patient in need thereof an amount of a RANK antagonist that is effective to stimulate new bone formation. The RANK antagonist preferably is a protein that is derived from the same species of animal as the patient. A “RANK agonist” is an agent that induces a biological activity associated with triggering RANK, such as inducing NF-κB activity. A “RANK antagonist,” as used herein, is an agent that blocks or reduces the interaction between RANK and RANKL, including agents that inhibit the synthesis of RANK or RANKL. RANK antagonists generally reduce biological activities associated with triggering RANK. In certain embodiments, the RANK antagonist comprises a soluble RANK or an antibody against RANK or RANKL that inhibits or blocks the interaction between RANK and RANKL and that does not stimulate a biological activity associated with triggering RANK. Another suitable RANK antagonist is OPG or soluble derivatives thereof, including dimers or higher level multimers. RANK antagonists typically will inhibit or block at least one of the biological activities associated with triggering RANK.

Triggering of RANK, such as by contact with membrane-bound or soluble RANKL or with an agonistic anti-RANK antibody, instigates RANK-mediated cellular responses that result from receptor oligomerization which may induce conformational changes in the cytoplasmic tail of the RANK protein. These cellular responses can include the activation of transcription factor NF-κB, a ubiquitous transcription factor that is extensively utilized in cells of the immune system, the activation of c-jun N-terminal kinase (JNK) or the activation of activator protein 1 (AP-1); see, for example, Galibert et al., J Biol Chem 273:34120-27 (1998) or Lee et al., Molec Pharmacol 55:1536-45 (2000). Triggering RANK in osteoclast progenitor cells induces the progenitors to differentiate into mature osteoclasts. RANK activation also enhances the bone-resorption activity of mature osteoclasts. Antagonists of RANK activity can be identified by virtue of their ability to inhibit or prevent any of the aforementioned manifestations of triggered RANK in a suitable assay, for example, in an assay that measures the biological activity of osteoclasts.

Assays may be conducted to determine whether a putative RANK antagonist is active in antagonizing RANK. The ability of a molecule to antagonize RANK can be readily determined, for example, in assays that measure the amount or activity of NF-κB in cells that express RANK, as described, for example, in U.S. Pat. No. 6,017,729, or that measure the amount or activity of JNK or AP-1, as described, for example, in Lee et al. (2000). In an assay for NF-κB, cells that express RANK are used, such as 293/EBNA cells. 293/EBNA cells are a cell line that was derived by transfection of the 293 cell line with a gene encoding Epstein-Barr virus nuclear antigen-1. To perform such an assay, 293/EBNA cells or other RANK-expressing test cells are exposed to a RANK trigger in the presence or absence of a putative RANK antagonist. The RANK trigger can be cells that express RANKL or soluble RANKL or an antibody that agonizes RANK activity. After exposure to the putative antagonist, the amount or activity of NF-κB in the triggered test cells is measured. If the putative antagonist inhibited the triggering of RANK, the amount or activity of NF-κB will not be elevated in the triggered test cells. If less NF-κB is detected in test cells exposed to the putative RANK antagonist than in cells not exposed to the molecule, then the molecule is determined to be a RANK antagonist. Alternatively, JNK or AP-1 activation can serve as a measure of RANK activity. Additional assays suitable for determining RANK antagonist activity include, for example, enzyme immunoassays or dot blots, assays that detect binding of labelled RANK to immobilized or cell-surface RANKL in the presence of increasing amounts of the fragment, or alternatively, assays that detect binding in the presence of the fragment of labelled RANKL to immobilized or cell-surface RANK. Such methods are well known in the art.

An exemplary nucleotide sequence encoding murine RANK is given in SEQ ID NO:1, and an exemplary nucleotide sequence encoding human RANK is given in SEQ ID NO:3; the corresponding full-length RANK polypeptides are shown, respectively, in SEQ ID NOS:2 and 4. Human RANK protein has 616 amino acid residues, while murine RANK has 625 amino acids, each comprising an extracellular domain capable of binding RANKL, a transmembrane region and a cytoplasmic domain. The cytoplasmic domain of RANK is capable of binding TRAFs 1, 2, 3, 5 and 6. The extracellular domain of human RANK corresponds to amino acids 1-213 of SEQ ID NO:4, and that of murine RANK to amino acids 1-214 of SEQ ID NO:2. The human RANK protein has a signal sequence that may be cleaved after any amino acid between residues 24 and 33 of SEQ ID NO:4, but which preferably is cleaved after amino acid 29. Murine RANK has a signal sequence that may be cleaved after any amino acid between residues 25 and 35 of SEQ ID NO:2, but that preferably is cleaved after amino acid 30.

The isolation of DNAs that encode human and murine RANK and RANKL are described in U.S. Pat. No. 6,017,729, which is incorporated by reference herein. RANK antagonists useful for practicing the invention include soluble RANK polypeptides capable of binding RANKL and that are encoded by nucleic acid molecules that are capable of hybridizing under stringent conditions to a nucleic acid (or its complement) that encodes a RANKL-binding portion of the extracellular region of a RANK protein such as that shown in SEQ ID NO:2 or NO:4. Such RANK antagonists may further comprise a heterologous signal peptide or the Fc region of an immunoglobulin or some other moiety to facilitate synthesis, purification or clinical efficacy of the protein when used as a therapeutic agent. Selection of appropriate hybridization conditions is well-known in the art, and a number of options are described, for example, see Sambrook et al. (Molecular Cloning: A Laboratory Manual, 2nd ed. (Cold Spring Harbor Laboratory, Cold Spring Harbor, N.Y.; 1989); pages 9.50-9.57 and 11.45-11.57, which are hereby incorporated by reference). Typically, when complex nucleic acids are used as labeled hybridization probes, they are fragmented prior to hybridization by treatment with alkali or mechanical shear to yield fragments varying from 50-600 nucleotides in length. For probes longer than about 50 nucleotides in length, stringent conditions are achieved by hybridizing at a temperature that is 20-25° C. below the melting temperature (Tm), while for oligonucleotide probes (typically 14-30 nucleotides in length), stringent conditions generally entail hybridizing at a temperature 5-10° C. below the melting temperature (see Sambrook et al., page 11.45). For probes greater than about 14 nucleotides in length, Tm can be calculated with reasonable accuracy using the formula Tm (° C.)=81.5+16.6(log₁₀[Na⁺])+0.41(% G+C)−(600/N), where N is the number of bases in the hybrid duplex, and [Na⁺] is the concentration of sodium ions in the hybridization buffer ([Na⁺] for 1×SSC=0.165M) (see Sambrook et al., page 11.46). If formamide is added to a hybridization solution, the Tm and therefore the optimal hybridization temperature becomes reduced by about 0.63° C. for each 1% formamide (Sambrook et al. at page 9.51). When a target nucleic acid is fixed to a solid support, stringent hybridization conditions may be achieved, for example, by hybridizing in 6×SSC at 63° C., and washing in 3×SSC at 55° C. Alternatively, stringent conditions can be achieved by hybridizing in 6×SSC plus 50% formamide at 42° C., followed by washing at room temperature (about 22° C.) in 2×SSC, then washing in 0.2×SSC at 68° C.

Exemplary nucleic acids that encode RANKL-binding soluble RANK polypeptides suitable for use as RANK antagonists include: a nucleic acid molecule encoding a polypeptide comprising amino acids x to y of SEQ ID NO:4, wherein x is selected from the group consisting of amino acids 1 to 33 of SEQ ID NO:4, and y is selected from the group consisting of amino acids 196 to 213 of SEQ ID NO:4; a nucleic acid molecule encoding a polypeptide comprising amino acids x to y of SEQ ID NO:2, wherein x is selected from the group consisting of amino acids 1 to 35 of SEQ ID NO:2, and y is selected from the group consisting of amino acids 197 to 214 of SEQ ID NO:2; and a nucleic acid molecule capable of hybridizing under stringent conditions with either of the foregoing nucleic acid molecules or its complement, in which the stringent conditions involve hybridizing in 6×SSC at 63° C., and washing in 3×SSC at 55° C.

In one embodiment of the invention, the nucleic acid molecule encoding a soluble RANK for use as a RANK antagonist in the subject invention will comprise nucleotides 91-642 of SEQ ID NO:1 (murine RANK) or nucleotides 126-677 of SEQ ID NO:3 (human RANK). The soluble RANK encoded by these nucleic acid molecules may correspond to any desired portion of a full-length RANK polypeptide so long as a sufficient amount of the RANK extracellular region is present to ensure binding to RANKL and the protein does not include the RANK transmembrane region.

In one aspect of the invention, patients in need thereof are treated by administering a RANK antagonist comprising a soluble RANK protein that is capable of binding RANKL and that comprises all or a fragment of the extracellular domain of a RANK protein. Soluble RANK may comprise the signal peptide and the extracellular domain of the exemplary human or murine RANK polypeptides disclosed herein, or, alternatively, the mature form of the protein with the signal peptide removed may be used.

In one aspect of the invention, soluble RANK polypeptides capable of binding RANKL are at least about 70% identical in amino acid sequence to the amino acid sequence of the extracellular region of native RANK protein as set forth in SEQ ID NOS:2 or 4 (respectively, amino acids 1-214 of SEQ ID NO:2 and amino acids 1-213 of SEQ ID NO:4). In another embodiment, the soluble RANK polypeptides bind RANKL and are at least about 80% identical in amino acid sequence to the extracellular region of a RANK polypeptide as shown in SEQ ID NOS:2 or 4. Also useful are RANK polypeptides that are capable of binding RANKL and that are at least about 90% identical to the RANKL-binding portion of the extracellular region of the native form of RANK as shown in SEQ ID NOS:2 or 4. Percent identity may be determined using a computer program, for example, the GAP computer program described by Devereux et al. (Nucl. Acids Res. 12:387, 1984) and available from the University of Wisconsin Genetics Computer Group (UTWGCG). For polypeptides encompassing fragments derived from the RANK protein, the identity is calculated based on that portion of the RANK protein that is present in the polypeptide. When the murine and human RANK proteins of SEQ ID NOS:2 and 4 are aligned as described here, they are found to be about 70% identical.

RANK polypeptides suitable for use as designed herein include polypeptides comprising amino acids 1-213 of SEQ ID NO:4 or amino acids 1-214 of SEQ ID NO:2 or alternatively may comprise RANKL-binding fragments thereof. If the patient is a human, the soluble RANK preferably is derived from a human RANK polypeptide. For human RANK, a polypeptide containing at least amino acids 33-196 of SEQ ID NO:4 can bind RANKL. One useful RANK antagonist is a polypeptide that comprises amino acids 30-213 of SEQ ID NO:4. If desired, a RANK antagonist comprising amino acids 30-213 of SEQ ID NO:4 may be fused to another protein that promotes dimerization.

RANK antagonists comprising a soluble RANK polypeptide may include other portions of RANK besides the extracellular domain but will not include the transmembrane region. The transmembrane regions of human and murine RANK are located, respectively, from about amino acid 214 to about amino acid 234 of SEQ ID NO:4 and from about amino acid 215 to about amino acid 235 of SEQ ID NO:2. Thus, soluble RANK antagonists suitable for the subject methods include proteins comprising, for example, a RANK extracellular region fused directly to a RANK intracellular region, such as a protein comprising amino acids 30-213 of SEQ ID NO:4 fused directly to a segment starting at about amino acid 235 and continuing through amino acid 616 of SEQ ID NO:4 or RANKL-binding portions thereof.

If desired, recombinant DNA techniques can be used to substitute a heterologous signal peptide for the native leader. A soluble RANK capable of binding RANKL may comprise a portion of human RANK having an amino terminus between amino acids 1 and 33 and continuing through amino acid 213 of SEQ ID NO:4. RANKL-binding fragments comprising portions of such a protein are useful as RANK antagonists and can be identified by various binding assays, such as those described herein. Alternatively, unique restriction sites or PCR techniques that are known in the art can be used to prepare nucleic acids encoding numerous truncated forms of RANK that can be expressed and analyzed for RANKL-binding activity.

RANKL-binding variants and alleles of RANK can be obtained using the methods and reagents provided in U.S. Pat. No. 6,017,729. The isolation of an allelic variant of human RANK has been reported which differs only slightly from the amino acid sequence shown in SEQ ID NO:4 (WO 98/54201). This variant of WO 98/54201, for example, has a valine instead of an alanine at the position corresponding to residue 192 of SEQ ID NO:4, and an isoleucine instead of a serine at the position corresponding to residue number 513 of SEQ ID NO:4. This RANK variant is capable of binding TRAFs and stimulating NF-κB and JNK. The human RANK proteins described in U.S. Pat. No. 6,017,729 or WO 98/54201 or any other RANKL-binding mutein or allelic variant of RANK may be used to derive soluble RANK proteins for use as antagonists in the subject invention. The ability of a RANK analog or mutein to be used to derive a soluble RANK for use as a RANK antagonist can be determined by testing the ability of the analogs or muteins to bind RANKL, for example as described in U.S. Pat. No. 6,017,729.

Also useful as therapeutic agents are soluble RANK proteins including covalent or aggregative conjugates of the proteins or their fragments with other proteins or polypeptides, such as by synthesis in recombinant culture as N-terminal or C-terminal fusions. For example, the conjugated peptide may be a signal (or leader) polypeptide sequence at the N-terminal region of the protein which co-translationally or post-translationally directs transfer of the protein from its site of synthesis to its site of function inside or outside of the cell membrane or wall (e.g., the yeast α-factor leader). Protein fusions can comprise peptides added to facilitate purification or identification of RANK proteins and homologs, such as poly(His). For example, a poly(His)₆ tag may be used (SEQ ID NO:6). The amino acid sequence of the inventive proteins can also be linked to an identification peptide such as that described by Hopp et al., Bio/Technology 6:1204, 1988 (FLAG™). This highly antigenic peptide provides an epitope reversibly bound by a specific monoclonal antibody, enabling rapid assay and facile purification of expressed recombinant protein. The sequence of Hopp et al. is also specifically cleaved by bovine mucosal enterokinase, allowing removal of the peptide from the purified protein.

In addition to soluble RANK, such fusion proteins may comprise, for example, a moiety such as an immunoglobulin Fc domain, a leucine zipper, polyethylene glycol or combinations thereof. Fusion proteins comprising RANKL-binding forms of soluble RANK suitable for use as described herein may be made using recombinant expression techniques. Such fusion proteins may form dimers or higher forms of multimers. Polymerized forms possess enhanced ability to inhibit RANK activity. Examples of fusion proteins that can polymerize include a RANK:Fc fusion protein, which can form dimers, and a fusion protein of a zipper moiety (i.e., a leucine zipper). Other useful fusion proteins may comprise various tags that are known in the art.

In one aspect of the invention, the antagonist is a fusion protein that comprises a soluble RANK linked to an immunoglobulin Fc region. If a human patient is being treated, the RANK and Fc moieties of the fusion protein preferably are derived from human sources. A useful Fc region for this purpose is one derived from a human IgG, immunoglobulin. Fragments of an Fc region may also be used, as can Fc muteins. For example, certain residues within the hinge region of an Fc region are critical for high affinity binding to Fc_{γ}RI. Canfield and Morrison (J. Exp. Med. 173:1483 (1991)) reported that Leu₍₂₃₄₎ and Leu₍₂₃₅₎were critical to high affinity binding of IgG₃ to Fc_{γ}RI present on U937 cells. Similar results were obtained by Lund et al. (J. Immunol. 147:2657 (1991); Molecular Immunol. 29:53 (1991)). Such mutations, alone or in combination, can be made in an IgG₁ Fc region to decrease the affinity of IgG₁ for FcR. Depending on the portion of the Fc region used, a fusion protein may be expressed as a dimer, through formation of interchain disulfide bonds. If the fusion proteins are made with both heavy and light chains of an antibody, it is possible to form a protein oligomer with as many as four RANK regions. A RANK:Fc fusion protein suitable for use in preparing a therapeutic composition is that shown in SEQ ID NO:5, which comprises the extracellular domain of a human RANK at amino acids 1-213 and an Fc region derived from a human IgGi immunoglobulin at amino acids 214-443. Amino acids 1-29 of SEQ ID NO:5 correspond to a leader sequence that may be cleaved off after the protein is translated in mammalian cells. An exemplary RANK:Fc fusion protein for use as a therapeutic agent is one consisting of an amino acid sequence as shown in SEQ ID NO:5, or one consisting of amino acids 30-443 of SEQ ID NO:5. In another embodiment of the invention, the RANK:Fc fusion protein used as a therapeutic agent is identical in sequence to amino acids 30-443 of SEQ ID NO:5 except that that glutamic acid is substituted for aspartic acid at residue 352 and methionine is substituted for leucine at residue 354.

Other soluble RANK proteins derivatives suitable for use as described herein comprise a soluble RANK polypeptide fused to an oligomerizing peptide such as a zipper domain. Leucine zippers were originally identified in several DNA-binding proteins and are present in the fos, jun and c-myc proteins (Landschulz et al., Science 240:1759 (1988)). “Zipper domain” is a term used to refer to a conserved peptide domain present in these (and other) proteins that is responsible for multimerization of the proteins. The zipper domain comprises a repetitive heptad repeat, with four or five leucine, isoleucine or valine residues interspersed with other amino acids. Examples of zipper domains are those found in the yeast transcription factor GCN4 and a heat-stable DNA-binding protein found in rat liver (C/EBP; Landschulz et al., Science 243:1681 (1989)). The products of the nuclear oncogenes fos and jun comprise zipper domains that preferentially form a heterodimer (O'Shea et al., Science 245:646 (1989); Turner and Tjian, Science 243:1689 (1989)). Zipper moieties useful for these purposes are described, for example, in U.S. Pat. No. 5,716,805.

In another aspect of the invention, the RANK antagonist is human OPG or a RANKL-binding derivative thereof. A nucleotide sequence encoding human OPG is shown in SEQ ID NO:7, and the corresponding amino acid sequence is shown in SEQ ID NO:8. OPG polypeptides suitable for use in the subject methods include those described in U.S. Pat. No. 6,369,027, which is hereby incorporated by reference in its entirety. OPG polypeptides useful as described herein include derivatives of the amino acid sequence shown in SEQ ID NO:8 that have an addition, deletion, insertion or substitution of one or more amino acids such that the polypeptide retains the ability to bind RANKL. For example, the OPG may have a deletion or carboxy-terminal truncation of all or part of amino acid residues 186-401 of SEQ ID NO:8; deletion of all or part of a cysteine-rich domain of OPG; and one or more amino acid changes in a cysteine-rich domain. In one embodiment, the OPG has from 1 to about 10 amino acids deleted from the mature amino terminus (located at amino acid residue 22), and, optionally, has from 1 to about 216 amino acids deleted from the carboxy terminus. By analyzing truncated forms of the protein, it has been shown that the biological activity of OPG is retained by a portion of OPG containing about 164 amino acids located at residues 22-185 of SEQ ID NO:8. Accordingly, an OPG polypeptide comprising amino acids 22-185 of SEQ ID NO:8 may be used to prepare therapeutic compositions that may be administered for the purposes described herein. Any of the aforedescribed OPG polypeptides may be fused with the Fc region of an immunoglobulin molecule. Full-length OPG spontaneously forms dimers or trimers, which are biologically active and may be administered for the subject methods.

Other RANK antagonists useful for the purposes described herein include small organic molecules.

In yet other embodiments of the invention, antagonists are used that have been designed to reduce the level of endogenous RANK or RANKL gene expression. Such antagonists are made using well-known antisense or ribozyme approaches to inhibit or prevent translation of RANK or RANKL mRNA transcripts; and triple helix approaches to inhibit transcription of RANK or RANKL genes. Techniques for the production and use of such molecules are well known to those of skill in the art.

Antisense RNA and DNA molecules useful as RANK antagonists can act to directly block the translation of MnRNA by hybridizing to targeted endogenous mRNA thereby preventing translation. This may be accomplished by using oligonucleotides (either DNA or RNA) that are complementary to RANK or RANKL mRNA, such as for example the anti-RANK antisense oligonucleotides described in U.S. Pat. No. 6,171,860. Useful antisense oligonucleotides include those that are complementary to the 5′ end of the mRNA, e.g., the 5′ untranslated sequence up to and including the AUG initiation codon. However, oligonucleotides complementary to the 5′- or 3′- non-translated, non-coding regions of the RANK or RANKL gene transcript, or to the coding regions, may be used. Antisense nucleic acids should be at least six nucleotides in length, and preferably are oligonucleotides ranging from 6 to about 50 nucleotides in length. The oligonucleotides can be DNA or RNA or chimeric mixtures or derivatives or modified versions thereof, single-stranded or double-stranded. Chimeric oligonucleotides, oligonucleosides, or mixed oligonucleotides/oligonucleosides of the invention can be of several different types. These include a first type wherein the “gap” segment of nucleotides is positioned between 5′ and 3′ “wing” segments of linked nucleosides and a second “open end” type wherein the “gap” segment is located at either the 3′ or the 5′ terminus of the oligomeric compound (see, e.g., U.S. Pat. No. 5,985,664). The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane or hybridization-triggered cleavage agents or intercalating agents.

For delivery to cells expressing RANK or RANKL, antisense DNA or RNA can be injected directly into the tissue or cell derivation site, or modified antisense molecules designed to target the desired cells (e.g., antisense linked to peptides or antibodies that specifically bind receptors or antigens expressed on the target cell surface) can be administered systemically. In one approach, target cells are transfected with a recombinant DNA construct in which the antisense oligonucleotide is placed under the control of a strong pol II or pol II promoter. Such a vector can remain episomal or become chromosomally integrated, as long as it can be transcribed to produce the desired antisense RNA. Vectors can be plasmid, viral, or others known in the art that are used for replication and expression in bacterial, yeast, insect or mammalian cells.

Ribozyme molecules designed to catalytically cleave RANK or RANKL mRNA transcripts can also be used to prevent translation of RANK or RANKL mRNA and expression of RANK or RANKL polypeptides. (See, e.g., WO 90/11364 or U.S. Pat. No. 5,824,519). The ribozymes that can be used to therapeutically antagonize RANK in the present invention include hammerhead ribozymes (Haseloff and Gerlach, 1988, Nature, 334:585-591), RNA endoribonucleases (hereinafter “Cech-type ribozymes”) such as the one which occurs naturally in Tetrahymena thermophila (known as the IVS, or L-19 IVS RNA) and which has been extensively described (see, for example, WO 88/04300; Been and Cech, Cell, 47:207-216 (1986)). Ribozymes can be composed of modified oligonucleotides (e.g. for improved stability, targeting, etc.) and should be delivered to cells which express the RANK or RANKL polypeptide in vivo. A preferred method of delivery involves using a DNA construct encoding the ribozyme under the control of a strong constitutive pol III or pol II promoter, so that transfected cells will produce sufficient quantities of the ribozyme to destroy endogenous RANK or RANKL messages and inhibit translation.

Other RANK antagonists useful as described herein include antibodies specific for RANK or RANKL. Antibodies used as RANK antagonists are specifically immunoreactive with their target, that is, they bind to the target protein via the antigen-binding site of the antibodies (as opposed to non-specific binding) and do not bind unrelated proteins to a significant degree. Thus, antibodies specific for RANK will bind RANK, but will not, for example, bind detectably to RANKL. Similarly, antibodies specific for RANKL will not bind detectably to RANK. Specifically binding antibodies will specifically recognize and bind a target RANK or RANKL polypeptide, such as those described herein, or subportions thereof, homologues, and variants thereof. Antagonistic antibodies specific for RANK or RANKL will bind endogenous RANK or RANKL, respectively, thus reducing the amount of endogenous target polypeptide available for binding to its respective cognate.

An antagonistic anti-RANK antibody, when bound with the extracellular domain of membrane-bound RANK, will not trigger RANK biological activity. For example, such an antibody thus will not induce an increase in NF-κB activity in RANK-expressing cells.

RANK antagonists suitable for use in the subject methods include antibodies that are specific for RANKL. Such antibodies can be prepared using the methods described herein or using other methods routine in the art. The nucleotide sequence of an exemplary nucleic acid encoding human RANKL is shown in SEQ ID NO:9, and the amino acid sequence encoded by this nucleotide sequence is shown in SEQ ID NO:10. Human RANKL contains a predicted 47 amino acid intracellular domain (corresponding to amino acids 1-47 of SEQ ID NO:10), a 21 amino acid transmembrane domain (corresponding to amino acids 48-68 of SEQ ID NO:10) and a 249 amino acid extracellular domain (corresponding to amino acids 69-317 of SEQ ID NO:10). The RANK-binding doman of human RANKL corresponds to about amino acid 162 to about amino acid 317 of SEQ ID NO:10. Purified polypeptides having the amino acid sequence of SEQ ID NO:10 or subportions thereof may be used to raise polyclonal or monoclonal antibodies that bind specifically with RANKL and block its ability to bind to RANK. RANKL polypeptides used to raise the subject antibodies may be fused, if desired, with another moiety, such as a leucine zipper, the Fc domain of an immunoglobulin, poly(His)₆, FLAG®, or other tag that may serve to facilitate synthesis or purification. In one aspect of the invention, the anti-RANKL antibodies are directed against epitopes present in a polypeptide comprising amino acids 69-317 of SEQ ID NO:10, which corresponds to the extracellular domain of human RANKL. The ability of a RANKL-specific antibody to block binding to RANKL can be determined by any convenient assay, such as those described in U.S. Pat. No. 6,242,2213, or any assay that measures a biological activity mediated by RANK-expressing cells that are exposed to RANKL.

In one aspect of the invention, RANKL polypeptides used to raise antibodies are, respectively, about 70% identical in amino acid sequence to the amino acid sequence of a RANKL protein described above, in another aspect of the invention they are about 80% identical, and in yet another aspect of the invention they are about 90% identical to the described RANKL polypeptides. Percent identity may be determined using a computer program, for example, the GAP computer program described by Devereux et al. (Nucl. Acids Res. 12:387, 1984) and available from the University of Wisconsin Genetics Computer Group (UWGCG). For polypeptides containing fragments derived from the RANKL protein, the percent identity is calculated based on that portion of the polypeptide that is derived from RANKL.

Also suitable for use as therapeutic agents of the subject invention are biologically active fragments of such antibodies. For example, a biologically active fragment of antibody is an antibody protein that is truncated relative to the intact antibody, but that retains the ability to specifically bind its target and to block that target's interaction with its cognate. Antigen-binding fragments of antibodies, include, but are not limited to, Fab and F(ab′)₂ fragments, and may be produced by conventional procedures.

Antibodies useful for therapeutic compositions according to the invention include but are not limited to polyclonal antibodies, monoclonal antibodies (mABs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Monoclonal antibodies to use as a RANK antagonist may be selected that are specific for epitopes present in human RANK or RANKL but not murine RANK or RANKL. Monoclonals that bind both mouse and human RANK or that bind both mouse and human RANKL also may be used as RANK antagonists for the subject therapeutic methods. Methods for obtaining monoclonal antibodies with a desired specificity are well known in the art, such as those described, for example, in U.S. Pat. No. 6,017,729. The RANK and RANKL polypeptides, fragments, variants and RANK fusion polypeptides as set forth herein can be employed as immunogens in producing antibodies specifically immunoreactive with RANK or RANKL.

### Synthesis of RANK Antagonists

RANK antagonists comprising a protein, such as purified soluble forms of RANK, OPG, antagonistic antibodies and homologs or analogs thereof are prepared by culturing suitable host/vector systems to express the recombinant translation products of the DNAs encoding the antagonist, which are then purified from culture media or cell extracts. A host cell that comprises an isolated nucleic acid of the invention, preferably operably linked to at least one expression control sequence, is a “recombinant host cell” and is said to be “transformed.” Monoclonal antibodies can be produced using standard procedures.

To recombinantly express a RANK antagonist that is a polypeptide, isolated nucleic acids encoding the antagonist can be operably linked to an expression control sequence such as the pDC409 vector (Giri et al., 1990, EMBO J., 13: 2821) or the derivative pDC412 vector (Wiley et al., 1995, Immunity 3: 673). The pDC400 series vectors are useful for transient mammalian expression systems, such as CV-1 or 293 cells. Alternatively, the isolated nucleic acid can be linked to expression vectors such as pDC312, pDC316, or pDC317 vectors. The pDC300 series vectors all contain the SV40 origin of replication, the CMV promoter, the adenovirus tripartite leader, and the SV40 polyA and termination signals, and are useful for stable mammalian expression systems, such as CHO cells or their derivatives. Alternatively, nucleic acids encoding the antagonist may be expressed using a vector having an internal polyadenylation signal, such as those described in WO 01/27299. Other expression control sequences and cloning technologies can also be used to produce the polypeptide recombinantly, such as the pMT2 or pED expression vectors (Kaufman et al., 1991, Nucleic Acids Res. 19:4485-4490; and Pouwels et al., 1985, Cloning Vectors: A Laboratory Manual, Elsevier, N.Y.) and the GATEWAY Vectors (Life Technologies; Rockville, Md.). In the GATEWAY system the isolated nucleic acid of the invention, flanked by attB sequences, can be recombined through an integrase reaction with a GATEWAY vector such as pDONR201 containing attP sequences. This provides an entry vector for the GATEWAY system containing the isolated nucleic acid of the invention. This entry vector can be further recombined with other suitably prepared expression control sequences, such as those of the pDC400 and pDC300 series described above. Many suitable expression control sequences are known in the art. General methods of expressing recombinant polypeptides are also described in R. Kaufman, Methods in Enzymology 185:537-566 (1990). As used herein, “operably linked” means that the nucleic acid of the invention and an expression control sequence are situated within a construct, vector, or cell in such a way that the polypeptide encoded by the nucleic acid is expressed when appropriate molecules (such as polymerases) are present. As one embodiment of the invention, at least one expression control sequence is operably linked to the nucleic acid of the invention in a recombinant host cell or progeny thereof, the nucleic acid and/or expression control sequence having been introduced into the host cell by transformation or transfection, for example, or by any other suitable method. As another embodiment of the invention, at least one expression control sequence is integrated into the genome of a recombinant host cell such that it is operably linked to a nucleic acid sequence encoding a polypeptide of the invention. In a further embodiment of the invention, at least one expression control sequence is operably linked to a nucleic acid of the invention through the action of a trans-acting factor such as a transcription factor, either in vitro or in a recombinant host cell.

A number of types of cells may act as suitable host cells for recombinant expression of polypeptides having RANK antagonist activity. Suitable mammalian host cells include, for example, the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, BHK (ATCC CRL 10) cell lines, the CV1/EBNA cell line derived from the African green monkey kidney cell line CVI (ATCC CCL 70) as described by McMahan et al. (EMBO J. 10: 2821, 1991), human kidney 293 cells, human epidermal A431 cells, human Colo205 cells, other transformed primate cell lines, normal diploid cells, cell strains derived from in vitro culture of primary tissue, primary explants, HL-60, U937, HaK or Jurkat cells. Alternatively, the polypeptide may be produced in lower eukaryotes such as yeast or in prokaryotes such as bacteria. Suitable yeast strains include Saccharomyces cerevisiae, Schizosaccharomyces pombe, Kluyveromyces strains, Candida spp., Pichia spp. or any yeast strain capable of expressing heterologous polypeptides. Potentially suitable bacterial strains include Escherichia coli, Bacillus subtilis, Salmonella typhimurium, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain a functional RANK antagonist. Such covalent attachments may be accomplished using known chemical or enzymatic methods.

The polypeptide may also be produced by operably linking the isolated nucleic acid of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), and Luckow and Summers, Bio/Technology 6:47 (1988).

Cell-free translation systems may also be employed to produce polypeptides using RNAs derived from nucleic acid constructs disclosed herein.

The polypeptide of the invention may be prepared by culturing transformed host cells under culture conditions suitable to support expression of the recombinant polypeptide. The resulting expressed polypeptide may then be purified from such culture (i.e., from culture medium or cell extracts) using known purification processes, such as selective precipitation with various salts, gel filtration and ion exchange chromatography. The purification of the polypeptide may also include an affinity column containing agents that will bind to the polypeptide; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography using an antibody that specifically binds one or more epitopes of the RANK antagonist.

To harvest the polypeptide RANK antagonist, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. For example, a suitable affinity matrix can comprise a counter structure protein or lectin or antibody molecule bound to a suitable support. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred. Gel filtration chromatography also provides a means of purifying the inventive proteins.

Affinity chromatography is a useful method of purifying RANK antagonists and homologs thereof. For example, a RANK expressed as a fusion protein comprising an immunoglobulin Fc region can be purified using Protein A or Protein G affinity chromatography. Moreover, a RANK protein comprising an oligomerizing zipper domain may be purified on a resin comprising an antibody specific to the oligomerizing zipper domain. Monoclonal antibodies against the RANK protein may also be useful in affinity chromatography purification, by utilizing methods that are well-known in the art. A ligand may also be used to prepare an affinity matrix for affinity purification of soluble RANK proteins or other RANK antagonists.

Finally, one or more reversed-phase high performance liquid chromatography (RP-HPLC) steps employing hydrophobic RP-HPLC media, e.g., silica gel having pendant methyl or other aliphatic groups, can be employed to further purify a RANK antagonist. Suitable methods include those analogous to the method disclosed by Urdal et al. (J. Chromatog. 296:171, 1984). Some or all of the foregoing purification steps, in various combinations, can also be employed to provide a homogeneous recombinant protein.

Recombinant protein produced in bacterial culture is usually isolated by initial extraction from cell pellets, followed by one or more concentration, salting-out, aqueous ion exchange or size exclusion chromatography steps. Finally, high performance liquid chromatography (HPLC) can be employed for final purification steps. Microbial cells employed in expression of recombinant protein can be disrupted by any convenient method, including freeze-thaw cycling, sonication, mechanical disruption, or use of cell lysing agents. Fermentation of yeast which express the inventive protein as a secreted protein greatly simplifies purification.

Protein synthesized in recombinant culture is characterized by the presence of cell components, including proteins, in amounts and of a character which depend upon the purification steps taken to recover the inventive protein from the culture. These components ordinarily will be of yeast, prokaryotic or non-human higher eukaryotic origin and preferably are present in innocuous contaminant quantities, on the order of less than about 1% by weight. Further, recombinant cell culture enables the production of the inventive proteins free of other proteins which may be normally associated with the proteins as they are found in nature in the species of origin.

### Therapeutic Methods

Provided herein are therapeutic methods for treating patients who require the formation of new bone, including but not limited to bone graft recipients, ligament graft recipients, prosthetic joint recipients, patients with a fractured bone, patients who have suffered a spinal cord injury, and patients who have completed a course of radiation treatment for cancer. The latter patients include those who are essentially cured of their cancer, that is, patients in whom no malignant tissue can be detected at the time treatment with a RANK antagonist is initiated. In one aspect of the invention, therapies are provided for patients who are not experiencing a loss of bone density at the time of treatment, yet who require the formation of new bone in order to repair earlier bone damage or to fill in gaps in bone. Other patients who will benefit from treatment with RANK antagonists include those suffering from conditions such as acute septic arthritis (including Reiter's syndrome), osteoarthritis, osteomalacias (including rickets and scurvy), hyperparathyroidism, Cushing's syndrome, polyostotic fibrous dysplasia, Gaucher's disease and Langerhans' cell histiocytosis. Suitable RANK antagonists for treating the above conditions include various soluble RANK polypeptides and antibodies specific for RANK or RANKL as described herein. Additional RANK antagonists that may be used include osteoprotegerin, ribozymes and antisense oligonucleotides.

Millions of patients each year require a bone graft. Among such patients are those with skeletal defects, including congenital defects, those who require joint fusions to stabilize damaged or unstable joints, and patients in whom a bone has been so badly damaged that some portion of the bone was lost and requires replacement. Bone graft recipients also include patients who have a gap in one or more bones due to revision joint surgery, excision of a bone tumor or oral/maxillofacial surgery. In one aspect of the invention, bone graft recipients include patients who are not experiencing abnormally high levels of osteoclast activity, that is, patients who are not undergoing an abnormal rate of bone loss at the time of treatment. Such patients include, for example, accident victims, patients who have been successfully treated for cancer and who no longer have cancer, persons undergoing voluntary bone reconstruction for cosmetic reasons, and persons undergoing surgery to correct skeletal defects. Patients with skeletal defects include, for example, patients with congenitally deformed bones, patients suffering from osteoarthritis and patients who have recovered from poliovirus infections.

For a bone graft, bone or synthetic material is shaped by the surgeon to fit the affected area, then held in place with pins or screws that hold the healthy bone to the implanted material. Host bone-forming cells will infiltrate the implant, which provides a structural framework to support the ingrowth of new bone, blood cells and soft tissue as they fill in the implant matrix and connect the graft to the host bone. A successful bone graft ultimately will exhibit a solid fusion of the infiltrated graft to the host bone to which the graft was adjoined. Both the filling in of the graft matrix and graft fusion to pre-existing host bone thus involves the formation of new bone. Accordingly, bone graft recipients will benefit from treatment with an agent that promotes new bone formation, such as a RANK antagonist provided herein.

Similarly, the anchoring of a prosthetic joint to host bone is a process that requires the formation of new bone, thus the subject therapeutic treatments are useful for treating patients who have undergone surgical implantation of a prosthetic joint. Prosthetic joints are often provided, for example, in patients with osteoarthritis, a condition characterized by degeneration of the articular cartilage and hypertrophy of bone at the margins and changes in the synovial membrane. About twenty percent of artificial joint recipients experience a gradual loosening of the prosthetic joint over the course of 20 years as a result of wear-debris osteolysis. However, the subject therapeutic methods are directed towards promoting integration and anchoring of a freshly implanted prosthetic device, rather than towards ameliorating the bone damage caused by the prosthetic loosening that occurs long after implant. The treatments provided herein are administered during or immediately following prosthesis placement. Prosthetic joint recipients treated in accord with the invention will receive a first dose of a RANK antagonist on the day of surgery, or within 1-6 days following prosthesis implant, or within 1-4 weeks following implant. The duration of such treatment will vary, but typically, repeated doses will be administered throughout the time the prosthesis is becoming attached to the patient's tissues, which process usually is complete within about 1-6 months following surgical implantation.

In one aspect of the invention, a RANK antagonist is administered to a patient who has received a bone graft in amounts and at a frequency of administration that is effective to promote the infiltration of the graft matrix and the solid fusion of the graft to the adjoining host bone. For a prosthetic joint recipient, the RANK antagonist is administered in amounts and at a frequency that is effective to promote attachment of the prosthesis to host bone and/or host tendons or ligaments. The RANK antagonist may be administered to such patients prior to, during or immediately following surgical implantation of the graft or prosthesis, or post-surgically at any time during the period in which graft infiltration and solid fusion or prosthesis attachment are taking place.

In addition, RANK; antagonists are used to enhance ligament attachment to bone in a patient who has undergone a ligament graft, including but not limited to patients who require a cruciate ligament graft following a knee injury. A successful grafted ligament will ultimately attach to the host bone, and such attachment requires the formation of new tissues, including new bone. Accordingly, ligament grafts may be treated by administering a RANK antagonist prior to, during or immediately following surgical implantation of the ligament graft, or post-surgically at any time during the period in which graft attachment to host bone is in process.

Bone graft or ligament graft recipients will receive a first dose of a RANK antagonist on the day of surgery, or within 1-6 days following graft implant, or within 1-4 weeks following graft implant. The duration of such treatment will vary, but typically repeated doses will be administered throughout the time the graft is undergoing infiltration and becoming attached to the patient's tissues. The infiltration/attachment process generally will be complete within about 1-6 months following the surgery.

The sufficiency of treatment for the above therapies may be monitored by the patient's physician by using physical examination or various radiographic methods, including ordinary x-rays, radiographic image enhancement, computed tomography (CT), magnetic resonance imaging (MRI), or by any other suitable means.

Periodontal reconstructive therapies can be used to repair periodontal osseous defects or periodontal injury or to enhance craniomaxillofacial surgery to the extent that it requires new bone formation. Moreover, bone grafting to repair an alveolar cleft has long been an integral part of the treatment of persons with unilateral and bilateral clefts of the lip and alveolus. Administration of the therapies described herein can promote periodontal reconstruction in patients in such patients.

Systemic bone loss often occurs following an injury to the spinal cord injury. This bone loss can contribute to the development of fragile bones that tend to fracture easily, including the bones of the hips or limbs. Such patients can benefit from treatment with an agent that promotes formation of new bone, such as the subject RANK antagonists. In one embodiment of the invention, a RANK antagonist is administered to a patient who has suffered a spinal cord injury in amounts and at a frequency of administration that is effective to stimulate the formation of new bone. The RANK antagonist may be administered to such patients immediately following the injury, or at any time thereafter, and may be administered in conjunction with physical therapy or other medications used to treat such injuries.

The invention further provides bone-restoring therapies for cancer patients who have suffered from bone loss following radiation therapy. Bone loss following radiation treatment occurs in the absence of tumor persistence, that is, it can occur even if the tumor has been successfully eliminated. This type of bone loss often is associated with radiation treatment of head and neck cancer, though it can occur with other types of cancer. Treatment of this condition involves administering a RANK antagonist after the course of radiation treatment is completed, and may be administered in conjunction with other treatments used to manage this condition, such as bone graft.

For the above therapeutic methods, the antagonist is administered in an amount and at a frequency effective to enhance the formation of new bone.

### Combination Therapies

The invention also contemplates the concurrent administration of RANK antagonists with various soluble cytokine receptors or cytokines, or other osteoclast/osteoblast regulatory molecules, or with other drugs used to treat patients who require restoration of lost bone. “Concurrent administration” encompasses simultaneous or sequential treatment with the components of the combination, as well as regimens in which the drugs are alternated, or wherein one component is administered long-term and the other(s) are administered intermittently. Such other drugs include, for example, bisphosphonates, or the use of more than one RANK antagonist administered concurrently. Examples of other drugs to be administered concurrently include but are not limited to antivirals, antibiotics, analgesics, corticosteroids, antagonists of inflammatory cytokines, DMARDs, various systemic chemotherapy regimens and non-steroidal anti-inflammatories, such as, for example, COX I or COX II inhibitors.

A useful combination comprises the concurrent administration of a RANK antagonist and an antagonist of TNFA, which is a cytokine associated with inflammatory responses. TNFα inhibitors alone may be used to treat any of the conditions described herein, or may be used concurrently with a RANK antagonist. TNFα inhibitors that may be used include, for example, soluble proteins comprising the extracellular region of a TNFα receptor (TNFR), which may be derived from TNFR I or II or other TNFRs. A useful TNFα inhibitor for these purposes is etanercept, which is a dimer of two molecules of the extracellular portion of the p75 TNFα receptor, each molecule consisting of a 235 amino acid TNFR-derived polypeptide that is fused to a 232 amino acid Fc portion of human IgG₁. Etanercept is currently sold by Immunex Corporation under the trade name ENBREL,® and generally is administered 1-3 times per week by subcutaneous injection at a flat dose of 25 or 50 mg/dose or at a dose of 5-12 mg/m². Other suitable TNFα inhibitors include antibodies against TNFα, including humanized antibodies. An exemplary humanized antibody for coadministration with a RANK inhibitor is infliximab (sold by Centocor as REMICADE®), which is a chimeric IgG1κ monoclonal antibody. Other suitable anti-TNFα antibodies include the humanized antibodies D2E7 and CDP571, and the antibodies described in EP 0 516 785 B1, U.S. Pat. No. 5,656,272, EP 0 492 448 A1. Additionally, TNFα may be inhibited by administering a TNFα-derived peptide that acts as a competitive inhibitor of TNFα (such as those described in U.S. Pat. No. 5,795,859 or U.S. Pat. No. 6,107,273), a TNFR-IgG fusion protein other than etanercept, such as one containing the extracellular portion of the p55 TNFα receptor, a soluble TNFR other than an IgG fusion protein, or other molecules that reduce endogenous TNFα levels, such as inhibitors of the TNFα converting enzyme (see e.g., U.S. Pat. No. 5,594,106), or small molecules such as pentoxifylline or thalidomide.

Similarly, inhibitors of the inflammatory cytokine IL-1 may be used alone to treat any of the conditions described above, or may be administered concurrently with a RANK antagonist. Suitable IL-1 inhibitors include, for example, receptor-binding peptide fragments of IL- 1, antibodies directed against IL-1, including IL-1α or IL-1β or other IL-1 family members, antagonistic antibodies against IL-1 receptor type I, and recombinant proteins comprising all or portions of receptors for IL-1 or modified variants thereof, including genetically-modified muteins, multimeric forms and sustained-release formulations. Other useful IL-1 antagonists include IL-1Ira polypeptides, IL-1β converting enzyme (ICE) inhibitors, IL-1 binding forms of type I IL-1 receptor and type II IL-1 receptor, and therapeutics known as IL-1 traps. IL-1ra polypeptides include the forms of IL-1ra described in U.S. Pat. No. 5,075,222 and modified forms and variants including those described in U.S. Pat. No. 5,922,573, WO 91/17184, WO 92 16221, and WO 96 09323. IL-1β converting enzyme (ICE) inhibitors include peptidyl and small molecule ICE inhibitors including those described in PCT patent applications WO 91/15577; WO 93/05071; WO 93/09135; WO 93/14777 and WO 93/16710; and EP 0 547 699. Non-peptidyl compounds include those described in WO 95/26958, U.S. Pat. No. 5,552,400, U.S. Pat. No. 6,121,266, and Dolle et al., J. Med. Chem. 39:2438-2440 (1996). Additional ICE inhibitors are described in U.S. Pat. Nos. 6,162,790, 6,204,261, 6,136,787, 6,103,711, 6,025,147, 6,008,217, 5,973,111, 5,874,424, 5,847,135, 5,843,904, 5,756,466, 5,656,627, 5,716,929. IL-1 binding forms of type I IL-1 receptor and type II IL-1 receptor are described in U.S. Pat. No. 4,968,607, U.S. Pat. No. 4,968,607, U.S. Pat. No. 5,081,228, Re U.S. Pat. No. 35,450, U.S. Pat. No. 5,319,071, and U.S. Pat. No. 5,350,683. IL-1 traps are described in WO 018932.

Further, suitable IL-1 antagonists encompass chimeric proteins that include portions of both an antibody molecule and an IL-1 antagonist molecule. Such chimeric molecules may form monomers, dimers or higher order multimers. Other suitable IL-1 antagonists include peptides derived from IL-1 that are capable of binding competitively to the IL-1 signaling receptor, IL-1 R type I.

Methods of the invention may utilize type II IL-1 receptor in a form that binds IL-1 and particularly IL-1β, and blocks IL-1 signal transduction, thereby interrupting the proinflammatory and immunoregulatory effects of IL-1, and particularly that of IL-1β. U.S. Pat. No. 5,350,683 describes type II IL-1 receptor polypeptide. Useful forms of the type II IL-1 receptor polypeptide include truncated soluble fragments that retain the capability of binding IL-1 and particularly IL-1β. Soluble type II IL-1 receptor molecules useful as IL-1 antagonists include, for example, analogs or fragments of native type II IL-1 receptor that lack the transmembrane region of the native molecule, and that are capable of binding IL-1, particularly IL-1βP.

Antagonists derived from type II IL-1 receptors (e.g. soluble forms that bind IL-1β) compete for IL-1 with IL-1 receptors on the cell surface, thus inhibiting IL-1 from binding to cells, thereby preventing it from manifesting its biological activities. Binding of soluble type II IL-1 receptor or fragments of IL-1 or IL-1β can be assayed using ELISA or any other convenient assay. If injected, the effective amount per adult dose of a soluble type II IL-1 receptor will range from 1-20 mg/r², and preferably will be about 5-12 mg/M². Alternatively, a flat dose may be administered, whose amount will range from 5-100 mg/dose, and more preferably will range from 20-50 mg/dose.

Soluble type II IL-1 receptor polypeptides or fragments suitable in the practice of this invention may be fused with a second polypeptide to form a chimeric protein. In one embodiment of such a chimeric protein, the second polypeptide may promote the spontaneous formation by the chimeric protein of a dimer, trimer or higher order multimer that is capable of binding IL-1 molecule and preventing it from binding to a cell-bound receptor that promotes IL-1 signaling. Chimeric proteins used as antagonists may be proteins that contain portions of both an antibody molecule and a soluble type II IL-1 receptor.

Furthermore, therapies administered in accord with the invention may be used in conjunction with local application to the affected bone of scaffolds of synthetic or natural biomaterials that promote the migration, proliferation, and differentiation of bone cells.

### Assays for Monitoring Bone Density

When administering the various treatments described herein, it is often convenient to monitor the sufficiency of treatment by measuring the patient's bone density at the onset of treatment and during the time treatment is being administered. Bone density is monitored in such a patient using standard techniques, including for example, single-photon absorptiometry, dual-photon absorptiometry, dual-energy x-ray absorptiometry, quantitative computed tomography and radiographic absorptiometry. A useful method of monitoring is dual-energy x-ray absorptiometry. Repeated bone density measurements of the same bone or bones permit the physician to estimate the rate of bone formation during treatment, thus enabling the physician to adjust the dose or frequency of administration of the RANK antagonist to optimize bone formation. An effective dosing regimen of a RANK antagonist, for example, will induce an increase in bone density of at least 2%, more preferably of at least 5%, and most preferably of at least 10% or more.

During the course of treatment, bone formation is expected to increase, thus the sufficiency of treatment may be monitored by repeatedly measuring bone density at any convenient interval. For example, density may be measured every week, every two weeks, every three weeks, every month, every three or more months or less often.

### Modes of Administration

For therapeutic use, a RANK antagonist is administered to an individual, preferably a human, for treatment in a manner appropriate to the indication. Systemic administration is generally appropriate for treating any indication requiring the generalized promotion of bone growth, such as, for example, when treating spinal cord injury patients or radiation therapy recipients. Alternatively, the RANK/RANKL antagonist may be applied locally, which may be appropriate for graft recipients, though these patients may be treated systemically if desired. For such patients, the RANK antagonist may if desired be applied directly to the graft implant at the time of surgery. Means of local administration include, for example, local injection, or application of the antagonist admixed or polymerized with a slow-release matrix suitable for this purpose, many of which are known.

This invention additionally provides for the use of RANK antagonists and drugs to be concurrently administered with RANK antagonists in the manufacture of a medicament for the treatment of numerous diseases. RANK antagonists and other drugs may be formulated into therapeutic compositions comprising an effective amount of the antagonist. In one embodiment of the invention, the therapeutic agent will be administered in the form of a pharmaceutical composition comprising a purified soluble protein having RANK antagonistic activity, in conjunction with physiologically acceptable carriers, excipients or diluents. Such carriers will be nontoxic to recipients at the dosages and concentrations employed. Inhibitors of the RANK/RANKL interaction for pharmaceutical compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Protein complexes with PEG can be made using known procedures, such as for example, those described in U.S. Pat. No. 5,849,860, U.S. Pat. No. 5,766,897 or other suitable methods. Suitable lipids for liposomal formulation include, without limitation, monoglycerides, diglycerides, cholesterol, sulfatides, lysolecithin, phospholipids, saponin, bile acids, and the like. Preparation of liposomal formulations is within the level of skill in the art, as disclosed, for example, in U.S. Pat. No. 4,235,871; U.S. Pat. No. 4,501,728; U.S. Pat. No. 4,837,028; U.S. Pat. No. 4,737,323; and U.S. Pat. No. 5,858,397. Such compositions will influence the physical state, solubility, stability, rate of in vivo release, and rate of in vivo clearance, and are thus chosen according to the intended application, so that the characteristics of the carrier will depend on the selected route of administration.

In one embodiment of the invention, sustained-release forms of RANK antagonists are used. Sustained-release forms suitable for use in the disclosed methods include, but are not limited to, soluble RANK polypeptides, osteoprotegerin and antagonistic anti-RANK or anti-RANKL antibodies that are encapsulated in a slowly-dissolving biocompatible polymer (such as the alginate microparticles described in U.S. Pat. No. 6,036,978), admixed with a slow-release polymer (including topically applied hydrogels), and/or incorporated into a biocompatible semi-permeable implant.

The amount of RANK antagonist administered per dose will vary depending on the antagonist being used and the mode of administration. If the antagonist is a soluble RANK and is administered by injection, the effective amount per adult dose will range from 0.5-20 mg/m², and preferably is about 5-12 mg/m². Alternatively, a flat dose may be administered, whose amount may range from 5-100 mg/dose. Exemplary dose ranges for a flat dose to be administered by subcutaneous injection are 5-25 mg/dose, 25-50 mg/dose and 50-100 mg/dose. The chosen dose may be administered repeatedly, particularly for chronic conditions, or the amount per dose may be increased or decreased as treatment progresses. For pediatric patients (ages 4-17), a suitable regimen involves the subcutaneous injection of 0.4 mg/kg, up to a maximum dose of 25 mg to be administered one or more times per week. If an antibody against RANK or RANKL is used as the RANK antagonist, preferred dose ranges include 0.1 to 20 mg/kg, 0.75 to 7.5 mg/kg and 1-10 mg/kg of body weight. Humanized antibodies are desirable, that is, antibodies in which only the antigen-binding portion of the antibody molecule is derived from a non-human source. Antibodies may be administered by injection, including intravenous infusion. Appropriate dosages can be determined in trials. The amount and frequency of administration will depend, of course, on such factors as the nature and severity of the indication being treated, the desired response, the condition of the patient, and so forth.

Ordinarily, the preparation of pharmaceutical compositions comprising a RANK antagonist entails combining the therapeutic protein with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with conspecific serum albumin are exemplary appropriate diluents. Preferably, product is formulated as a lyophilizate using appropriate excipient solutions (e.g., sterile water or sucrose solution) as diluents. One embodiment of the invention entails packaging a lyophilized RANK antagonist in dose unit form which when reconstituted will provide one to three doses per package.

The compounds of the present invention may be administered orally, parenterally, sublingually, by inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. Injection is a preferred route of administration, including parenteral injection. Parenteral injections include subcutaneous injections, intraspinal, intrathecal, intraorbital, intravenous, intrarterial, intramuscular, intrasternal, and infusion techniques. Compositions comprising a RANK antagonist can be administered by bolus injection or continuous infusion. Useful routes of systemic administration are subcutaneous injection and intravenous drip.

In other embodiments of the invention, cells genetically modified to express a RANK antagonist are employed. For example, DNA encoding a soluble RANK or other protein with RANK antagonist activity is introduced into cells removed from the patient's body, and the cells thereafter returned to the patient. The DNA is introduced in a form that promotes expression of the antagonist in the recipient cells, that is, the coding regions are operably linked to appropriate regulatory elements for expression in the cells. The DNA may be introduced using a suitable vector, such as a retroviral or adenovirus vector, or encapsulated in liposomes. Suitable cells for this mode of drug administration include cells that will home to the affected tissue, such as bone marrow cells, including hematopoietic progenitor cells. In other similar embodiments, cell lines are modified to express the antagonist by introduction of DNA encoding the RANK antagonist, then the cells are introduced into the patient. Such cells may be transformed with DNA constructs that promote either stable or transient expression of the RANK antagonist. Alternatively, DNA encoding the antagonist may be introduced into the patient encapsulated in liposomes, which may be administered systemically or locally into the affected tissues.

Various animal models of the diseases to be treated are known in the art; accordingly, one can apply routine experimentation to determine optimal dosages and routes of administration of the RANK antagonist, first in an animal model and then in human patients. Optimally, the dosing regimen will be adjusted so that the newly formed bone is of high quality and resembles normal bone. The specific dosing regimen for any particular patient will depend upon a variety of factors including the activity of the specific compound employed, the age, body weight, general health, sex, diet, time of administration, route of administration, rate of excretion, drug combination, and the severity of the patient's condition. It is expected that the patient's physician will adjust the dose and frequency of administration as needed to obtain optimal results.

What is claimed is:

1. A method of treating a patient in need thereof comprising administering to said patient a RANK antagonist in an amount and at a frequency sufficient to stimulate an increase in the rate of formation of new bone, wherein:
(a) said RANK antagonist is capable of inhibiting the ability of RANK to induce NF-κB, wherein RANK is a proteinconsisting of amino acids 1-616 of SEQ ID NO:4;
(b) said patient is selected from the group consisting of patients who have acute septic arthritis, osteomalacia, hyperparathyroidism, Cushing's syndrome, monoostotic fibrous dysplasia, polyostotic fibrous dysplasia, Gaucher's disease, Langerhans' cell histiocytosis, spinal cord injury, patients requiring periodontal reconstruction and patients who have completed a course of radiation therapy for cancer; and
(c) said RANK antagonist is selected from the group consisting of an antibody capable of specifically binding a RANK polypeptide comprising amino acids 33-196 of SEQ ID NO:4; an antisense oligonucleotide that blocks transcription or translation of RANK or RANKL mRNA; a ribozyme that cleaves RANK or RANKL mRNA; an antibody capable of specifically binding a RANKL polypeptide comprising amino acids 69-317 of SEQ ID NO:10; and an OPG polypeptide that comprises amino acids 22-185 of SEQ ID NO:8 and is capable of binding a RANKL polypeptide consisting of amino acids 1-317 of SEQ ID NO:10.

2. A method according to claim 1, wherein the patient has not experienced loss of bone density for at least one month preceding the initiation of treatment.

3. A method according to claim 1, wherein the sufficiency of treatment is monitored by repeatedly measuring bone density during the time the treatment is being administered.

4. A method according to claim 1, wherein the RANK antagonist is an antibody capable of specifically binding a RANKL polypeptide comprising amino acids 69-317 of SEQ ID NO:10.

5. A method according to claim 1, wherein the RANK antagonist is an OPG polypeptide that comprises amino acids 22-185 of SEQ ID NO:8 and is capable of binding a RANKL polypeptide consisting of amino acids 1-317 of SEQ ID NO:10.

6. A method of treating a patient in need thereof comprising administering to said patient a RANK antagonist in an amount and at a frequency sufficient to stimulate an increase in the rate of formation of new bone, wherein:
(a) said RANK antagonist is capable of inhibiting the ability of RANK to induce NF-κB, wherein RANK is a protein consisting of amino acids 1-616 of SEQ ID NO:4;
(b) the RANK antagonist is a soluble RANK polypeptide that is capable of binding a RANKL polypeptide comprising amino acids 69-317 of SEQ ID NO:10, wherein said soluble RANK polypeptide has an at least 90% identity to a RANK polypeptide comprising amino acids 33-196 of SEQ ID NO:4; and
(c) said patient is selected from the group consisting of patients who have acute septic arthritis, osteomalacia, hyperparathyroidism, Cushing's syndrome, monoostotic fibrous dysplasia, polyostotic fibrous dysplasia, Gaucher's disease, Langerhans' cell histiocytosis, spinal cord injury, patients requiring periodontal reconstruction and patients who have completed a course of radiation therapy for cancer.

7. A method according to claim 6, wherein said soluble RANK polypeptide is encoded by a nucleic acid molecule that is capable of hybridizing under stringent conditions with a nucleic acid molecule consisting of the nucleotide sequence shown in SEQ ID NO:3 or its complement, wherein said stringent conditions comprise hybridizing in 6×SSC at 63° C., and washing in 3×SSC at 55° C.

8. A method according to claim 6, wherein the soluble RANK polypeptide comprises amino acids 33-196 of SEQ ID NO:10.

9. A method according to claim 6, wherein the soluble RANK polypeptide further comprises a moiety selected from the group consisting of an immunoglobulin Fc domain, a FLAG™ tag, a peptide comprising at least about 6 His residues, a leucine zipper, polyethylene glycol and combinations thereof.

10. A method according to claim 9, wherein the further moiety comprises an immunoglobulin Fc domain.

11. A method according to claim 10, wherein the soluble RANK polypeptide consists of amino acids 30-433 of SEQ ID NO:5.

12. A method according to claim 10, wherein the soluble RANK polypeptide consists of amino acids 30-433 of SEQ ID NO:5 except that glutamic acid is substituted for aspartic acid at residue 352 and methionine is substituted for leucine at residue 354.

13. A method according to claim 6, wherein the patient has not experienced loss of bone density for at least one month preceding the initiation of treatment.

14. A method according to claim 6, wherein the sufficiency of treatment is monitored by repeatedly measuring bone density during the time the treatment is being administered.

15. A method of treating a human patient in need thereof comprising administering to said patient a RANK antagonist in an amount and at a frequency sufficient to stimulate an increase in the rate of formation of new bone, wherein:
(a) the RANK antagonist is selected from the group consisting of a soluble RANK polypeptide that is capable of binding a RANKL polypeptide comprising amino acids 69-317 of SEQ ID NO:10, said soluble RANK polypeptide having an at least 90% identity to a RANK polypeptide comprising amino acids 33-196 of SEQ ID NO:4; an antibody capable of specifically binding a RANK polypeptide comprising amino acids 33-196 of SEQ ID NO:4; an antisense oligonucleotide that blocks transcription or translation of RANK or RANKL mRNA; a ribozyme that cleaves RANK or RANKL mRNA; an antibody capable of specifically binding a RANKL polypeptide comprising amino acids 69-317 of SEQ ID NO:10; and an OPG polypeptide that comprises amino acids 22-185 of SEQ ID NO:8 and is capable of binding a RANKL polypeptide consisting of amino acids 1-317 of SEQ ID NO:10;
(b) said patient is selected from the group consisting of prosthetic joint recipients, bone graft recipients and ligament graft recipients; and
(c) the first dose of the antagonist is administered within one month of surgical implantation of the prosthetic joint, bone graft or ligament graft.

16. A method according to claim 15, wherein the sufficiency of treatment is monitored by repeatedly measuring bone density during the time the treatment is being administered.

17. A method according to claim 15, wherein the RANK antagonist is an antibody capable of specifically binding a RANKL polypeptide comprising amino acids 69-317 of SEQ ID NO:10.

18. A method according to claim 15, wherein the RANK antagonist is an OPG polypeptide that comprises amino acids 22-185 of SEQ ID NO:8 and is capable of binding a RANKL polypeptide consisting of amino acids 1-317 of SEQ ID NO:10.

19. A method according to claim 15, wherein the RANK antagonist is a soluble RANK polypeptide comprising amino acids 33-196 of SEQ ID NO:4.

20. A method according to claim 19, wherein the soluble RANK polypeptide further comprises a moiety selected from the group consisting of an immunoglobulin Fc domain, a FLAG™ tag, a peptide comprising at least about 6 His residues, a leucine zipper, polyethylene glycol and combinations thereof.

21. A method according to claim 20, wherein the soluble RANK polypeptide consists of the amino acid sequence shown in SEQ ID NO:5.

22. A method according to claim 20, wherein the soluble RANK polypeptide consists of amino acids 30-433 of SEQ ID NO:5 except that glutamic acid is substituted for asparatic acid at residue 352 and methionine is substituted for leucine at residue 354.
